(19) United States
(12) Patent Application Publication
(10) Pub. No.: US 2007/0239061 A1
(43) Pub. Date: Oct. 11, 2007

(54) Systems and methods for providing multi-variable measurement diagnostic

(76) Inventors: Jacob Carter, Pleasant Grove, UT (US); Jonathan G. Wilcock, Orem, UT (US); Douglas S. Horne, Murray, UT (US); Valentine Krzyzaniak, South Federal Way, WA (US)

Correspondence Address: KIRTON AND MCCONKIE, 60 EAST SOUTH TEMPLE,, SUITE 1800, SALT LAKE CITY, UT, 84111, US

(21) Appl. No.: 11/398,373

(22) Filed: Apr. 5, 2006

(51) Int. Cl. A61B 5/05; A61H 39/02

(52) U.S. Cl. 600/547; 600/548

(57) ABSTRACT

The present invention relates to accurately measuring electrical readings. One embodiment of the present invention includes a method for measuring the electrical readings, recording the electrical readings to produce a relationship, correlating the relationship to a plurality of known mistakes, and then generating an accuracy level. The relationship may be a mathematical or graphical relationship among the measured electrical readings. The relationship can then be correlated to relationships that are produced when a particular measurement error is made. The probability that particular measurement errors were made can be determined such that an overall accuracy level can be quantified. In addition, the method may include suggesting corrective actions if the accuracy level indicates a high probability of measurement error. Another embodiment of the present invention includes a system for accurately measuring electrical readings. The system includes modules for receiving the electrical readings, correlating the relationship of the readings to known measurement mistakes, and outputting an accuracy level.

### BACKGROUND OF THE INVENTION

1. Field of the Invention

The present invention relates to treatment and diagnosis methods. More particularly, the present invention relates to a multi-variable measurement diagnostic for evaluating and correcting measurements.

2. Background and Related Art

Traditional medical science has long recognized certain electrical characteristics of humans and other living organisms. For example, the traditional medical community has recognized electrical potentials generated by the human body in such forms as brain waves, detected by electro-encephalographs (EEG), electrical impulses resulting from muscular heart activity, as detected -by electrocardiograms (EKG), and other electrical potentials measurable at other areas of the human body. While the levels of electrical activity at sites on the human body are relatively small, such signals are nonetheless measurable and consistent across the species.

In addition to measurable currents, the human body and other mammalian organisms exhibit specific locations where a resistance value and, inversely, a conductance value are relatively predictable for healthy individuals. These locations, known as anatomical dermal conductance points, exhibit unique resistance values. Interestingly, such locations exhibit a resistive reading of approximately 100,000 ohms and coincide with the acupuncture points defined anciently by the Chinese.

Ancient Chinese medical practitioners treated many unfavorable health conditions by inserting thin needles into the body at specific points to pierce peripheral nerves, a technique commonly known as acupuncture. Acupressure is a gentle, noninvasive form of the ancient Chinese practice of acupuncture that implements thumb or finger pressure or electrical stimulation at these same points, also known as acupressure points, to provide similar results.

The representative acupressure points and their relationship with organs and life systems of the human body have been characterized into more than 800 points that are organized into approximately 12 basic meridians that run along each side of the body. Each pair of meridians corresponds to a specific organ or function such as stomach, liver, spleen/pancreas and lung. Acupressure points are named for the meridian they lie on, and each is given a number according to where along the meridian it falls. For example, Spleen 6 is the sixth point on the Spleen meridian. The measurable attributes of each acupressure point reflect the energetic condition of the inner organ or other functions of the human body corresponding to such point.

Acupressure points are generally located at the extremity region of the hands and feet. As introduced above, the resistance value of healthy tissue measured at an acupressure point is generally in the range of about 100,000 ohms. When conditions arise affecting higher electrical readings, perhaps from inflammation or infection, the measured resistance value becomes less than 100,000 ohms. Likewise when conditions arise affecting lower electrical readings, perhaps from tissue fatigue or a degenerative state, conductivity is reduced, causing the resistance value to be higher.

Systems have been implemented to measure a resistance, voltage, and/or current values at acupressure points located on a meridian and to present the values to a clinician for use in assessing a condition. Unfortunately, these measurements are often inconsistent or inaccurate. For example, one practitioner may apply more pressure than another causing an inaccurate reading. Likewise, a reading taken from an inappropriate location will also cause an inaccurate reading. Conventional systems have attempted to correct these common mistakes with proper training and education of practitioners. However, this does not guarantee accurate results. Therefore, a system is required that can analyze existing readings and diagnose the likelihood that a mistake was made in the measurement of the readings.

### SUMMARY OF THE INVENTION

The present invention relates to accurately measuring electrical readings. One embodiment of the present invention includes a method for measuring the electrical readings, recording the electrical readings to produce a relationship, correlating the relationship to a plurality of known mistakes, and then generating an accuracy level. The relationship may be a mathematical or graphical relationship among the measured electrical readings. The relationship can then be correlated to relationships that are produced when a particular measurement error is made. The probability that particular measurement errors were made can be determined such that an overall accuracy level can be quantified. In addition, the method may include suggesting corrective actions if the accuracy level indicates a high probability of measurement error. Another embodiment of the present invention includes a system for accurately measuring electrical readings. The system includes modules for receiving the electrical readings, correlating the relationship of the readings to known measurement mistakes, and outputting an accuracy level.

These, and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a suitable operating environment for the present invention;

FIG. 2 illustrates a flow chart of one embodiment of the present invention; and

FIG. 3 illustrates a system schematic of an alternative embodiment of the present invention.

### DETAILED DESCRIPTION OF THE INVENTION

The present invention relates to accurately measuring electrical readings. One embodiment of the present invention includes a method for measuring the electrical readings, recording the electrical readings to produce a relationship, correlating the relationship to a plurality of known mistakes, and then generating an accuracy level. The relationship may be a mathematical or graphical relationship among the measured electrical readings. The relationship can then be correlated to relationships that are produced when a particular measurement error is made. The probability that particular measurement errors were made can be determined such that an overall accuracy level can be quantified. In addition, the method may include suggesting corrective actions if the accuracy level indicates a high probability of measurement error. Another embodiment of the present invention includes a system for accurately measuring electrical readings. The system includes modules for receiving the electrical readings, correlating the relationship of the readings to known measurement mistakes, and outputting an accuracy level. While embodiments of the present invention are directed at medical and homeopathic applications, it will be appreciated that the teachings of the present invention are applicable to other fields.

As used in this specification, the following terms are defined accordingly:

“electrical readings”—electrical readings on one or more locations of the human body including but not limited to resistance, capacitance, inductance, etc.

“relationship”—some form of relationship between multiple data objects including mathematical, graphical, visual, etc.

“measurement mistakes”—mistakes made in measurement which cause a reading to be inaccurate.

“correlation”—mathematically matching or comparing data.

“accuracy”—a value corresponding to how likely a measurement was made correctly.

The following disclosure of the present invention is grouped into two subheadings, namely “Exemplary Operating Environment” and “Multi-Variable Measurement Diagnostic”. The utilization of the subheadings is for convenience of the reader only and is not to be construed as limiting in any sense.

### Exemplary Operating Environment

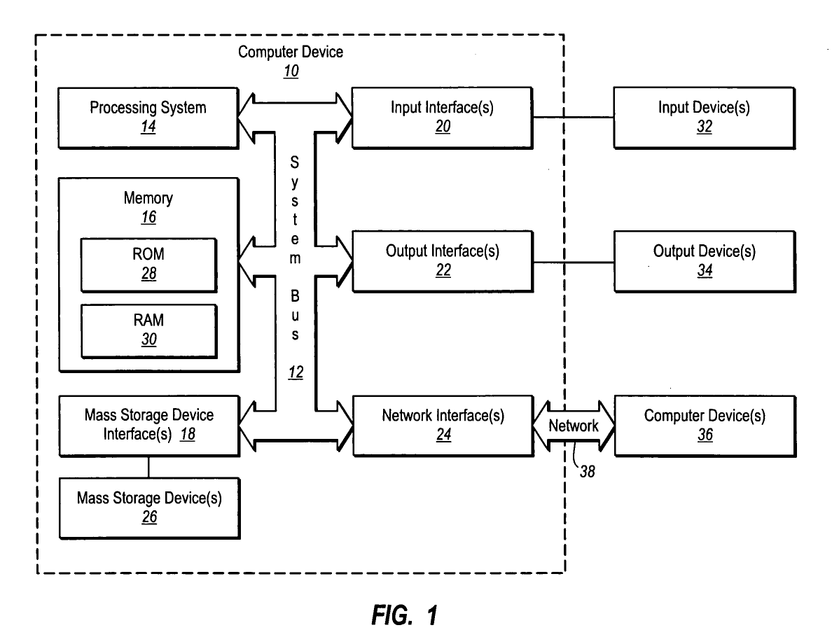

FIG. 1 and the corresponding discussion are intended to provide a general description of a suitable operating environment in which the invention may be implemented. One skilled in the art will appreciate that the invention may be practiced by one or more computing devices and in a variety of system configurations, including in a networked configuration. Alternatively, the invention may also be practiced in whole or in part manually following the same procedures.

Embodiments of the present invention embrace one or more computer readable media, wherein each medium may be configured to include or includes thereon data or computer executable instructions for manipulating data. The computer executable instructions include data structures, objects, programs, routines, or other program modules that may be accessed by a processing system, such as one associated with a general-purpose computer capable of performing various different functions or one associated with a special-purpose computer capable of performing a limited number of functions. Computer executable instructions cause the processing system to perform a particular function or group of functions and are examples of program code means for implementing steps for methods disclosed herein. Furthermore, a particular sequence of the executable instructions provides an example of corresponding acts that may be used to implement such steps. Examples of computer readable media include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing system.

With reference to FIG. 1, a representative system for implementing the invention includes computer device 10, which may be a general-purpose or special-purpose computer. For example, computer device 10 may be a personal computer, a notebook computer, a personal digital assistant (“PDA”) or other hand-held device, a workstation, a minicomputer, a mainframe, a supercomputer, a multi-processor system, a network computer, a processor-based consumer electronic device, or the like.

Computer device 10 includes system bus 12, which may be configured to connect various components thereof and enables data to be exchanged between two or more components. System bus 12 may include one of a variety of bus structures including a memory bus or memory controller, a peripheral bus, or a local bus that uses any of a variety of bus architectures. Typical components connected by system bus 12 include processing system 14 and memory 16. Other components may include one or more mass storage device interfaces 18, input interfaces 20, output interfaces 22, and/or network interfaces 24, each of which will be discussed below.

Processing system 14 includes one or more processors, such as a central processor and optionally one or more other processors designed to perform a particular function or task. It is typically processing system 14 that executes the instructions provided on computer readable media, such as on memory 16, a magnetic hard disk, a removable magnetic disk, a magnetic cassette, an optical disk, or from a communication connection, which may also be viewed as a computer readable medium.

Memory 16 includes one or more computer readable media that may be configured to include or includes thereon data or instructions for manipulating data, and may be accessed by processing system 14 through system bus 12. Memory 16 may include, for example, ROM 28, used to permanently store information, and/or RAM 30, used to temporarily store information. ROM 28 may include a basic input/output system (“BIOS”) having one or more routines that are used to establish communication, such as during start-up of computer device 10. RAM 30 may include one or more program modules, such as one or more operating systems, application programs, and/or program data.

One or more mass storage device interfaces 18 may be used to connect one or more mass storage devices 26 to system bus 12. The mass storage devices 26 may be incorporated into or may be peripheral to computer device 10 and allow computer device 10 to retain large amounts of data. Optionally, one or more of the mass storage devices 26 may be removable from computer device 10. Examples of mass storage devices include hard disk drives, magnetic disk drives, tape drives and optical disk drives. A mass storage device 26 may read from and/or write to a magnetic hard disk, a removable magnetic disk, a magnetic cassette, an optical disk, or another computer readable medium. Mass storage devices 26 and their corresponding computer readable media provide nonvolatile storage of data and/or executable instructions that may include one or more program modules such as an operating system, one or more application programs, other program modules, or program data. Such executable instructions are examples of program code means for implementing steps for methods disclosed herein.

One or more input interfaces 20 may be employed to enable a user to enter data and/or instructions to computer device 10 through one or more corresponding input devices 32. Examples of such input devices include a keyboard and alternate input devices, such as a mouse, trackball, light pen, stylus, or other pointing device, a microphone, a joystick, a game pad, a satellite dish, a scanner, a camcorder, a digital camera, and the like. Similarly, examples of input interfaces 20 that may be used to connect the input devices 32 to the system bus 12 include a serial port, a parallel port, a game port, a universal serial bus (“USB”), a firewire (IEEE 1394), or another interface.

One or more output interfaces 22 may be employed to connect one or more corresponding output devices 34 to system bus 12. Examples of output devices include a monitor or display screen, a speaker, a printer, and the like. A particular output device 34 may be integrated with or peripheral to computer device 10. Examples of output interfaces include a video adapter, an audio adapter, a parallel port, and the like.

One or more network interfaces 24 enable computer device 10 to exchange information with one or more other local or remote computer devices, illustrated as computer devices 36, via a network 38 that may include hardwired and/or wireless links. Examples of network interfaces include a network adapter for connection to a local area network (“LAN”) or a modem, wireless link, or other adapter for connection to a wide area network (“WAN”), such as the Internet. The network interface 24 may be incorporated with or peripheral to computer device 10. In a networked system, accessible program modules or portions thereof may be stored in a remote memory storage device. Furthermore, in a networked system computer device 10 may participate in a distributed computing environment, where functions or tasks are performed by a plurality of networked computer devices.

### Multi-Variable Measurement Diagnostic

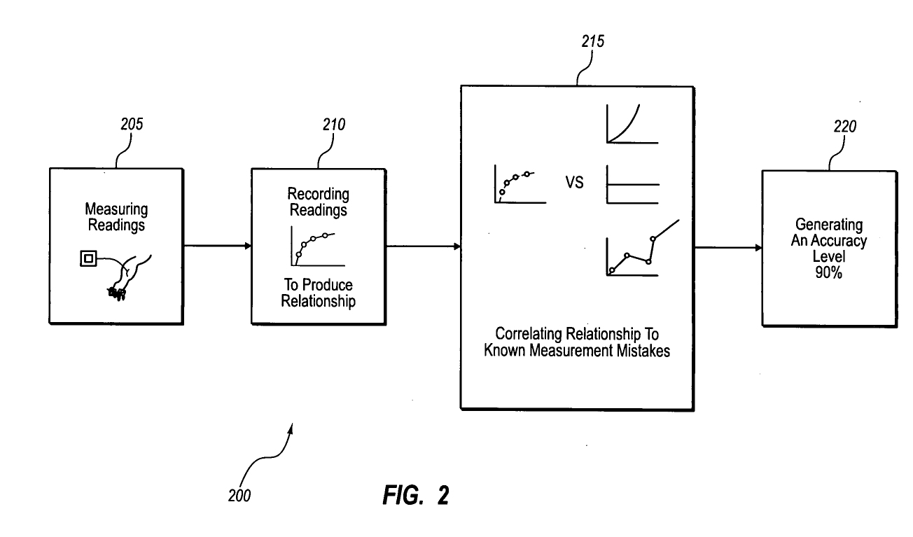

Reference is next made to FIG. 2, which illustrates flow chart of one embodiment of the present invention, designated generally at 200. The illustrated embodiment is a method for accurately measuring electrical readings. Initially, the electrical readings are measured, act 205. This act may be performed manually or automatically depending on the device or equipment used for measurement. For example, a machine may be used to automatically make the measurement to increase the reliability of the measurement. The electrical readings are often measured at locations corresponding to the known acupuncture points.

The electrical reading are then recorded, act 210. The act of recording the electrical readings may involve an operator manually writing the measured readings. Alternatively, the act of recording may include recording measured or received data from an input device. For example, if the measurements were taken manually, a user may enter the values of the measurements into a computer via a keyboard. Likewise, if the measurements were taken with a device or machine, the device will transfer the information to a location to be recorded. The recorded readings naturally have a relationship to one another. This relationship may include a mathematical relationship or a graphical relationship.

The relationship between the electrical readings is then correlated with known relationships produced by known mistakes, act 215. This involves a multi-variable comparison of the current relationship to other known relationships that result from measurement mistakes. The similarity between the relationship and the known relationships is an indication of whether a particular measurement mistake was made.

Based on the correlation, an overall accuracy level is able to be determined, act 220. The accuracy level is an indication corresponding to the probability that a mistake was made in measuring the electrical readings. This accuracy level is also mathematically related to the similarity between the relationship and one or more of the relationships produced as a result of known measurement mistakes.

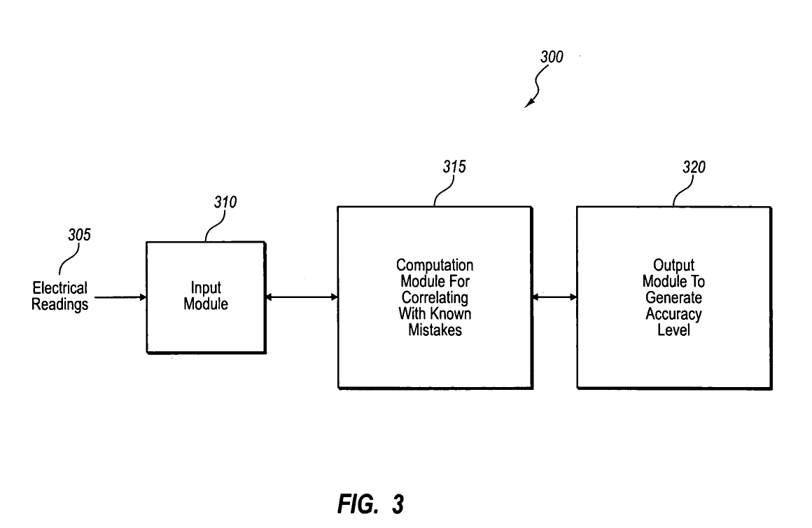

Reference is next made to FIG. 3, which illustrates a system schematic of an alternative embodiment of the present invention, designated generally at 300. The system 300 is capable of determining the accuracy that a set of electrical readings were properly measured. The system includes an input module 310 for receiving the electrical readings 305. The input module 310 may receive the electrical readings directly or indirectly from a device or operator. The input module 310 transmits the measured electrical readings to a computation module 315. The computation module performs at least one form of correlation or comparison algorithm to compare the relationship among the electrical readings to relationships produced as a result of known measurement mistakes. The computation module transmits data related to the results of its algorithms to an output module 320. The output module computes and/or displays an overall accuracy level that is an indication of the likelihood that a measurement error occurred in the measurement of the electrical readings.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A method for accurately measuring electrical readings, comprising the acts of:
measuring at least two electrical readings for different regions of the human body;
recording the at least two electrical regions to produce a relationship therebetween;
correlating the relationship to a plurality of known relationships that correspond to specific measurement mistakes; and
generating an accuracy level corresponding to the likelihood that a mistake was made in measuring the at least two electrical readings.

2. The method of claim 1, wherein the act of measuring at least two electrical readings further includes measuring the impedance from a particular location on the human body to a second location on the human body.

3. The method of claim 1, wherein the act of measuring at least two electrical readings further includes measuring the resistance from a particular location on the human body to a second location on the human body.

4. The method of claim 1, wherein the at least two electrical readings include a plurality of electrical readings corresponding to the acupuncture points on the human body.

5. The method of claim 1, wherein the act of measuring at least two electrical readings for different regions of the human body includes using a measurement device.

6. The method of claim 1, wherein the act of recording the at least two electrical regions to produce a relationship therebetween further includes charting the at least two measurements on a common axis.

7. The method of claim 6, wherein the act of correlating the relationship to a plurality of known relationships that correspond to specific measurement mistakes further includes comparing the appearance of the graph to the appearance of similar measurement graphs that correspond to situations in which a particular mistake was made during measurement.

8. The method of claim 1, wherein the act of correlating the relationship to a plurality of known relationships that correspond to specific measurement mistakes further includes:
mathematically correlating the relationship to individual relationships that correspond to measurement mistakes;
calculating a probability for known each measurement mistake; and
calculating a total probability for all known measurement mistakes.

9. The method of claim 1, wherein the measurement mistakes include inappropriate pressure during measurement.

10. The method of claim 1, wherein the measurement mistakes include excess moisture present on measurement surface.

11. The method of claim 1, wherein the measurement mistakes includes inaccurate location of measurement.

12. The method of claim 1, wherein the act of generating an accuracy level corresponding to the likelihood that a mistake was made in measuring the at least two electrical readings further includes:
displaying an accuracy level; and
if the accuracy level indicates a substantial likelihood of a measurement mistake, suggesting correction procedures to correct the at least two measurements.

13. The method of claim 12, wherein the correction procedures correspond to particular known measurement mistakes.

14. The method of claim 12, wherein the accuracy level includes an overall accuracy level and a plurality of individual likelihoods that particular measurement mistakes were made.

15. A method for accurately measuring electrical readings, comprising the acts of:
measuring at least two electrical readings for different regions of the human body;
recording and charting the at least two electrical regions to produce a charted relationship therebetween;
correlating the charted relationship to a plurality of known relationships that correspond to specific measurement mistakes; and
generating an accuracy level corresponding to the likelihood that a mistake was made in measuring the at least two electrical readings;
if the accuracy level indicates a substantial probability that a mistake was made, suggesting correction procedures to correct the at least two measurements.

16. A system for accurately measuring electrical readings, comprising:
an input module for receiving at least two electrical readings for different regions of the human body;
a computation module for correlating a relationship among the electrical readings to a plurality of known relationships that correspond to specific measurement mistakes; and
an output module that generates an accuracy level corresponding to the likelihood that a mistake was made in measuring the at least two electrical readings.

17. The system of claim 16, wherein the input module, computation module, and output module are disposed within the same device.

18. The system of claim 16, wherein the computation module is disposed remotely and connected via a data connection.

19. The system of claim 16, wherein the input module is coupled to a measurement device such that the measurements are automatically recorded.
